(19) United States
(12) Patent Application Publication
(10) Pub. No.: US 2022/0287734 A1
(43) Pub. Date: Sep. 15, 2022

(54) NOSE CLEANING SYSTEM

(71) Applicants: Emee Reburiano TAN, Manila (PH); John Balatbat ECHAUZ, Quezon City (PH); Gabriel Romeo Echauz LICHAUCO, Muntinlupa City (PH); Maria Carmen Salandanan TAN, Manila (PH); Alexander Chan YU, Portland, OR (US)

(72) Inventors: Emee Reburiano TAN, Manila (PH); John Balatbat ECHAUZ, Quezon City (PH); Gabriel Romeo Echauz LICHAUCO, Muntinlupa City (PH); Maria Carmen Salandanan TAN, Manila (PH); Alexander Chan YU, Portland, OR (US)

(21) Appl. No.: 17/200,035

(22) Filed: Mar. 12, 2021

(51) Int. Cl. A61B 17/24

(52) U.S. Cl. CPC A61B 17/24; A61B 2017/246; A61M 2210/0618

(57) ABSTRACT

A nose cleaning system for personal care and hygiene for the nose includes a nose cleaner having a cleaning head that has a textured surface; a neck that is wider than the cleaning head; a gripping body that is of smaller circumference (or perimeter) than the neck, and ergonomically designed to sit well in the fingers; a handle base; and a cover that is connectable to the neck; and a foaming nose cleanser having a pH of about 6. The foaming nose cleanser includes water (90%); a mix of synthetic detergents (6%); humectants or moisture enhancers (1.5%), salt (1.5%); and additives (1%).

### BACKGROUND OF THE INVENTION

### Field of the Invention

The invention relates to personal hygiene particular to instruments, components and methods of cleaning the nose.

### The Relevant Technology

Nose picking is a common habit or reflex often viewed as socially unacceptable. It is a personal necessity due to a buildup of dry or moist mucus along with various dirt in the nose from the usual dust and debris to airborne occupational hazards, which herein will be collectively referred to as nasal elements. While saline rinse administered via spray or irrigation mechanism is commonly prescribed for cleansing the nasal cavities especially for allergy and sinusitis sufferers, nose cleaning is mostly relegated to the fingers. Even children's instructional literature would teach kids alternative methods like using tissues and wet wipes followed by handwashing. However, variations in anatomical dimensions can sometimes make the use of fingers or finger-assisted methods (in the case of tissue and wipes) not feasible. Furthermore, such practices could pose risks from micro cuts and minor irritation to spreading bacteria and viruses from fingers to the nasal cavity and from fingers to the proximal environment. A reliable, safe, effective and hygienic means of cleaning the nose is needed.

In the prior art, mechanisms for cleaning the nose refer to swabbing of mucus, and scraping or scooping of debris. These cleaning methods relate to devices such as swabs with filamentous, fibrous or absorbent properties that collect mucus in the nose, and cotton bud-like implements with dual ends to scrape debris or ear pick-like instruments with a cleaning tip or loop. The cleaning heads or tips of the devices described in prior art vary from cotton bud-shaped swabs to hook-shaped scrapers. U.S. Pat. No. 9,532,793 issued to Wilson in 2017 describes a disposable nostril cleaning device of various parts that includes a cleaning head assembly with an option for various attachments like a cotton swab and a scraper tip. U.S. Pat. Application No. 20080300527 describes a handheld and disposable device for manually cleaning body orifices with ridges situated on the cleaning tip. The patent further suggests the option of imparting herbal scents like tea tree, eucalyptus and peppermint to the cleaning tips. Similarly, U.S. Pat. Application No. 20190209390 describes a nose cleaning swab system comprising of an auger swab head with swirled profile that removes materials out of a bodily orifice like the nose by twisting the device. Such twisting motion and rotational or circular movement of a cleaning device in the nose are also important to the cleaning mechanism of the present invention. An added feature of the present nose cleaning system pertains to the cleaning head that is designed with an embossed surface mimicking the light texture imparted by a handkerchief or a tissue, which will allow for a gentle, safe and effective physical mechanism for washing away nasal elements. The inventors would also like to note that ridges and swirled assemblies on the swab head are likely to irritate the soft tissues of the nasal cavity.

Other inventions introduce reusability of the nose cleaning apparatus without the need for intensive sanitation of the devices. U.S. Pat. No. 8,241,236 issued to Yardley/Y B Innovations, LLC in 2012 describes a modular apparatus for cleaning nasal cavities. In said invention, an antimicrobial cloth or similar pliable material attaches to the head of the apparatus, which is designed to be inserted to the nostril for facilitation of the removal of debris. The present invention similarly offers reusability, but without having a modular design, providing an advantage of longer reusability as the unibody design presented is easy to clean using the same foaming cleanser for use for cleaning the nose and does not have crevices or similar conducive spaces where bacteria and other organisms can thrive. In the present invention, the foaming nose cleanser is a specially formulated cleanser composed of a mix of synthetic detergents, among others, intended for use as foam and dispensed using a foaming pump. Consequently, the nose cleanser, as one of the subjects of the present invention, may alternately be referred to as foaming nose cleanser, foaming cleanser or nose cleanser foam in this document.

U.S. Pat. No. 5,895,408 issued to Pagan in 1999 describes a soft-bodied device for cleaning the internal walls and hairs of the nose that can be impregnated with a cleansing compound. While the idea of a cleansing compound like eucalyptus, camphor and menthol for cleaning and refreshing the nose was introduced, the role of said compound in cleaning the nose was not described. In the present invention, a foaming nose cleanser with pH compatible to the nose is proposed to be dispensed on the nose cleaner, which is inserted into the nostril to clean the nose followed by rinsing with tap water. A key characteristic of the foaming cleanser introduced in the current invention is the pH that corresponds to the naturally and slightly acidic environment of the nasal mucous membranes. In addition, the foaming nasal cleanser can have compounds that impart a refreshing and clean feeling to the user, among other cosmetic sensibilities.

### SUMMARY OF THE INVENTION

The aforementioned advantages of the novel nose cleaning system addresses the deficiencies in prior art as further clarified by the object of the present invention.

It is the main objective of the present invention to provide a hygienic means of cleaning the nose that will render nose picking a thing of the past, replacing the fingers with a sanitary device that effectively cleans the nose. The nose cleaner is designed to be used with a nose-compatible foaming cleanser and tap water for rinsing. This practice encourages the user to fulfill personal necessities relating to the nose much like tooth brushing, that is, within the confines and convenience of a bathroom at home or elsewhere. Through this system, nose cleaning can be habituated to daily hygiene practices like tooth brushing and bathing, allowing perhaps the discontinuation of mindless nose picking. Nose cleaning is seen to especially benefit populations residing in areas with poor air quality or individuals susceptible to inhaling pollutants or elements that easily collect or accumulate in the nose.

A further object of the invention is to ensure safety and efficacy in nose cleaning. According to one embodiment of the present invention, the nose cleaner comprises of a cleaning head with an embossed texture that provides enough friction or traction to dislodge nasal elements with the aid of the nose cleanser foam, and yet gentle enough not to leave micro cuts commonly imparted when fingernails hit the soft tissues of the nose. In the same embodiment, the cleaning head connects to the neck that has a wider width (from a two-dimensional perspective) ensuring that the cleaning tip does not reach farther than the anterior nasal cavity, and irritate nor injure the mucous membranes.

It is another object of the invention to consider environmental and economic aspects in the design. The nose cleaner has a unibody construct that is finitely reusable, and preferably produced using a single type of polymer or material that can be easily manufactured and recycled at its end-of-life. In addition, the present invention introduces a novel system of cleaning the nose that does not use tissue or any single-use fibrous and absorbent material that ends up in the trash as infectious waste. The present system proposes a wash and rinse method of nose cleaning that requires water only for rinsing the cleanser from the nasal cavity. The use of a foam nose cleanser is proposed in this method for water efficiency and effective cleaning. Dispensing and using a nose cleanser in the form of a foam does not require wetting the nose prior to cleaning it, and only necessitates minimal water use for rinsing the nose as only a sufficient amount of foaming cleanser is used. The choice of using a foaming pump to dispense the nose cleanser allows for a fixed or predetermined amount of foam to be dispensed that is adequate for the purpose.

The aforementioned objects of the invention along with the novel features, functionalities and advantages will become more apparent with reference to the illustrations and detailed descriptions that follow. The drawings only present one embodiment of the invention, and the invention may be embodied in many different forms, sizes and shapes in accordance with the salient design principles claimed herein.

### BRIEF DESCRIPTION OF THE DRAWINGS

FIG. 1 illustrates a perspective view of nose cleaner, and

FIG. 2 illustrates a diagonal angle view of the nose cleaner.

### DETAILED DESCRIPTION OF PREFERRED EMBODIMENT

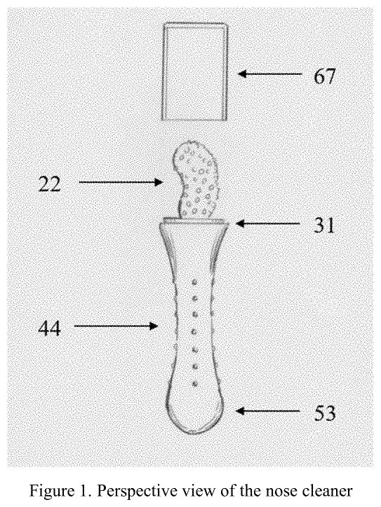

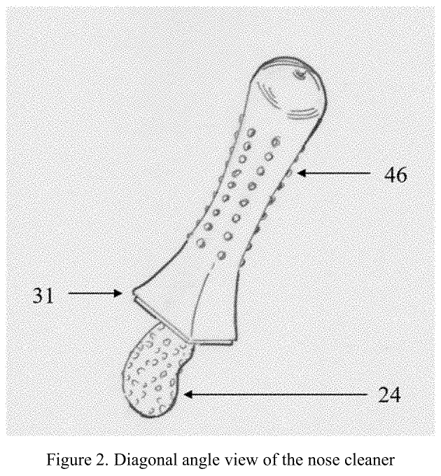

The above objects of the invention and the accompanying drawings of an embodiment of the invention to be read with the detailed description will better exemplify the various aspects and cleaning mechanism of the present invention. The nose cleaner device 10 depicted in FIGS. 1 and 2 are provided for illustrative purposes.

The nose cleaner 10 in FIG. 1 is a unibody device composed of a cleaning head 22, a neck 31, gripping body 44, handle base 53 and cover 67. While the cleaning head 22 may have symmetrical and asymmetrical sides in the embodiment provided, the key features of the shape are the broad tip of the cleaning head and smooth outline of the structure that tapers to the neck 31 of the nose cleaner. Cleaning head 22 also presents with both concave and convex sides to facilitate removal of nasal elements. The stubby tip of the cleaning head 22 offers a secondary safety mechanism to the neck 31 by signaling to the user an endpoint much like a finger will when used for nose picking, allowing the user to palpate the point up in the nose at which cleaning is intended and will not cause harm to surrounding structures. The neck 31 offers the primary safety mechanism in acting as a stopper by preventing the device from being pushed way up the delicate nasal structures. The neck 31 is defined by a larger width than the cleaning head 22 at its widest and also larger circumference than the nostril of the intended user whether a child or an adult. The lightly textured outline of the cleaning head 22 is also designed with safety in mind and in particular consideration for the mucous membranes and inner walls of the nose, which are densely lined with delicate nerve endings susceptible to scratches and cuts.

In the same embodiment in FIG. 1, the gripping body 44 is smaller in circumference than the perimeter of the neck 31, and gradually flutes to the handle base 53 allowing for it to be held by fingers. As a handheld and manually operated device, ergonomic design of the gripping body 44 makes it easier for the fingers and wrist to maneuver the nose cleaner 10 in the nose without much effort or strain. The handle base 53, as presented in FIG. 1, has a spherical presentation that goes well with the angular neck 31 for horizontal storage or stability when placed on a flat surface. In other embodiments, neck 31 can have a rounded circumference and an angular handle base 53 to provide the same stability and prevent the nose cleaner from rolling when placed flat. Similarly, the handle base 53 can have an angular structure and a flat bottom, or a suction cup bottom for upright storage. The square cover 67 in the present embodiment fits on the neck of the device for convenient storage. The cover 67 complements the preferred design and shape of the neck 31 for proper fit.

As illustrated in FIGS. 1 and 2, nose cleaner 10 has a unibody construction for ease of manufacture, use, storage and recycling. The nose cleaner 10 is designed to be used with a nose cleanser formulated at a pH of about 6 to ensure compatibility with the naturally slightly acidic environment of the nasal cavity. The nose cleanser is prepared as liquid, but proposed to be dispensed as foam for use in cleaning the nose through the action of a foaming pump. For this purpose, the inventors find the following formulation of the nose cleanser to produce a velvety foam and effective nose cleaning action: water (90%); synthetic detergents, particularly anionic and zwitterionic surfactants (6%); humectants or moisture enhancers (1.5%); salt (1.5%); and additives (1%) such as stabilizers, preservatives and fragrances. Fragrances and plant essential oils can be an added option in the formulation of the nose cleanser to impart a fresh scent among others.

In the present nose cleaning system, the nose cleanser foam is dispensed onto the cleaning head 22 of nose cleaner 10 for use in cleaning the anterior nasal cavity of elements. The cleaning head 22 is inserted in the nostril along with the nose cleanser foam. The neck 31 ensures that the cleaning head does not overreach and injure the sensitive structures up the nasal passage. The texture offered by the embossed surface 46 of the gripping body 44 allows for the nose cleaner to be maneuvered within the anterior nasal cavity through a circular or rotational motion with ease. The nasal elements are effectively loosened through the combination of the cleaning action of the foaming nose cleanser and the cleaning head 22. The foam and the cleaning head 22 are meant to capture or loosen the nasal elements. On contact with the anterior nasal cavity, some of the foam disintegrates as it cleans the nasal walls and can be easily rinsed away with tap water by cupping hands to the nose. After which, the nose cleaner can be cleaned using the same foaming cleanser and rinsed before storage.

The embossed surface 46 of the gripping body 44 can take on various patterns and shapes to create a roughened surface for better grip. In FIG. 2, the embossed surface 46 is shown to take the form of spherical textures lining the gripping body 44 horizontally. Likewise, the surface of the cleaning head 22 is shown to have an embossed texture 24 for superior cleaning action. This embossed texture 24 may be randomly situated or form a pattern on the entire surface of the cleaning head 22, providing a bit of non-irritating abrasiveness, similar to the texture offered by fibrous materials or cloth, critical to the cleaning action of the device. The embossed texture 24 is shown to take a circular shape in FIG. 2.

The foregoing descriptions of the embodiment present an exemplary illustration of the novelty and functionality of the nose cleaning system. Various interpretations and embodiments may be made of the principles and concepts of the described invention. Dimensions of the nose cleaner 10 and respective parts were not specified in account of anatomical size variations with age and gender. In the tests conducted by the inventors among adult subjects, for example, the cleaning head 22 of nose cleaner 10 finds a suitable proportion at a height of not more than 25 mm, and width of not more than 13 mm at its widest; the neck 31, wider than the width of the cleaning head, at 21 mm; the gripping body 44 at a height of 65 mm and width at 12 mm at its narrowest; and the spherical handle base 53 at 17 mm at its diameter; and with the cover 67 on, the nose cleaner measures at 100 mm in height. The described embodiment is not to be seen as a restrictive interpretation of the essence of the invention.

1. A nose cleaning system for personal care and hygiene for the nose, comprising:
a nose cleaner having:
a cleaning head that has a textured surface;
a neck that is wider than the cleaning head;
a gripping body that is of smaller circumference (or perimeter) than the neck, and ergonomically designed to sit well in fingers;
a handle base; and
a cover that is connectable to the neck; and
a foaming nose cleanser having a pH of about 6, and comprising:
water (90%);
a mix of synthetic detergents (6%);
humectants or moisture enhancers (1.5%);
salt (1.5%); and
additives (1%).

2. A method of using the nose cleaning system in claim 1, comprising the steps of:
engaging the nasal elements in the anterior nasal cavity with the nose cleaner and foaming nose cleanser;
rotating the cleaning head in the anterior nasal cavity to loosen the elements; and
rinsing the anterior nasal cavity with water by cupping the hands to the nose.
